(19) United States
(12) Patent Application Publication
(10) Pub. No.: US 2023/0172934 A1
(43) Pub. Date: Jun. 8, 2023

(54) NOVEL EXTENDED RELEASE COMPOSITION OF TOFACITINIB, ITS DERIVATIVES AND SALTS

(71) Applicant: Zim Laboratories Limited, Maharashtra (IN)

(72) Inventors: Anwar DAUD, Maharashtra (IN); Chandrashekhar MAINDE, Maharashtra (IN); Uttam KEDAR, Maharashtra (IN); Kuldeep GANGAWAT, Maharashtra (IN)

(21) Appl. No.: 17/922,921

(22) PCT Filed: May 17, 2021
(86) PCT No.: PCT/IB2021/054195

§ 371 Date: Nov. 2, 2022

(30) Foreign Application Priority Data
May 18, 2020 (IN) 202021020825

(51) Int. Cl. A61K 31/519; A61K 9/28; A61K 9/20

(52) U.S. Cl. CPC A61K 31/519; A61K 9/2853; A61K 9/2018; A61K 9/2027; A61K 9/2031; A61K 9/2054; A61K 9/2013

(57) ABSTRACT

The present invention relates to solid composition of Tofacitinib and salt thereof, and process of manufacture thereof. The present invention relates to non-osmotic an extended release composition comprising Tofacitinib or salt thereof, mixture of polyethylene oxides along with one or more pharmaceutically acceptable excipient. The non-osmotic an extended release composition of Tofacitinib or salt thereof is used in the treatment of Rheumatoid Arthritis, Psoriatic Arthritis and Ulcerative Colitis.

### FIELD OF THE INVENTION

The present invention relates to extended release composition of Tofacitinib or salt thereof, and process of manufacture thereof. Specifically the present invention relates to the extended release composition of Tofacitinib or salt thereof comprising mixture of polyethylene oxide.

### BACKGROUND OF THE INVENTION

Rheumatoid arthritis is an autoimmune and inflammatory disease, which means that your immune system attacks healthy cells in your body by mistake, causing inflammation (painful swelling) in the affected parts of the body. Rheumatoid arthritis mainly attacks the joints, usually many joints at once. Rheumatoid arthritis commonly affects joints in the hands, wrists, and knees. In a joint with Rheumatoid arthritis, the lining of the joint becomes inflamed, causing damage to joint tissue. This tissue damage can cause long-lasting or chronic pain, unsteadiness (lack of balance), and deformity (misshapenness). The signs and symptoms associated with Rheumatoid arthritis are Pain or aching in more than one joint, Stiffness in more than one joint, Tenderness and swelling in more than one joint, Weight loss, Fever, Fatigue or tiredness and Weakness. There is no cure for rheumatoid arthritis. But clinical studies indicate that remission of symptoms is more likely when treatment begins early with medications known as disease-modifying antirheumatic drugs (DMARDs). The types of medications include NSAIDs, Steroids, Disease-modifying antirheumatic drugs (DMARDs) include methotrexate, leflunomide, hydroxychloroquine, sulfasalazine and biologic response modifiers include abatacept, adalimumab, anakinra, baricitinib, certolizumab, etanercept, golimumab, infliximab, rituximab, sarilumab, tocilizumab and tofacitinib.

Psoriatic Arthritis is a form of arthritis that affects some people who have the skin condition psoriasis. Psoriatic arthritis is a type of inflammatory arthritis. Symptoms include joint pain, stiffness and swelling, which may flare and subside. Many people with the condition are affected by morning stiffness. Even mild skin psoriasis can have a significant degree of arthritis.

The management of Rheumatoid arthritis and Psoriatic Arthritis include treatment with medications like methotrexate, leflunomide, hydroxychloroquine, sulfasalazine and biologic response modifiers include abatacept, adalimumab, anakinra, baricitinib, certolizumab, etanercept, golimumab, infliximab, rituximab, sarilumab, tocilizumab and tofacitinib.

Ulcerative colitis is an inflammatory bowel disease (IBD) that causes inflammation and ulcers (sores) in your digestive tract. Ulcerative colitis affects the innermost lining of your large intestine (colon) and rectum. Symptoms usually develop over time, rather than suddenly. Signs and symptoms associated with Ulcerative colitis are Diarrhea, often with blood or pus, Abdominal pain and cramping, Rectal pain, Rectal bleeding—passing small amount of blood with stool, Urgency to defecate Inability to defecate despite urgency, Weight loss, Fatigue, Fever, In children, failure to grow. The management of ulcerative colitis include use of Anti-inflammatory drugs like 5-aminosalicylates, Corticosteroids. Immunosuppressant drugs like Azathioprine, mercaptopurine, Cyclosporine, Tofacitinib. Biologics like Infliximab, adalimumab, golimumab, Vedolizumab, Ustekinumab. Other medications include Anti-diarrheal medications, Pain relievers, Antispasmodics and Iron supplements.

Tofacitinib is Janus kinase (JAK) inhibitor. The chemical name of Tofacitinib citrate is (3R, 4R)-4-methyl-3-(methyl-7H-pyrrolo [2,3-d]pyrimidin-4-ylamino)-ß-oxo-1-piperidinepropanenitrile, 2-hydroxy-1, 2, 3-propanetricarboxylate (1:1). Its empirical formula is C₁₆H₂₀N₆O.C₆H₈O₇ and molecular weight is 504.5 Daltons. Tofacitinib citrate is represented by structural formula (I)

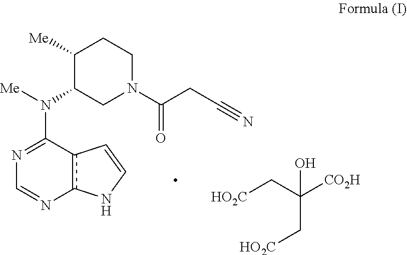

Tofacitinib citrate is a white to off-white powder. The solubility of tofacitinib citrate in water is 2.9 mg/ml.

Tofacitinib citrate Extended release tablet [11 mg & 22 mg] is approved in USA under the brand name XELJANZ XR. Tofacitinib citrate Extended release tablet [11 mg] is approved in UK under the brand name XELJANZ. The product is indicated for the treatment of Rheumatoid Arthritis, Psoriatic Arthritis and Ulcerative Colitis

Tofacitinib citrate Extended release tablet under the trade name XELJANZ XR Contain 11 mg/22 mg of Tofacitinib and inactive ingredient like cellulose acetate, copovidone, hydroxyethyl cellulose, hydroxyl propylcellulose, HPMC 2910/Hypromellose, magnesium stearate, red iron oxide, sorbitol, titanium dioxide and triacetin. Printing ink contains, ammonium hydroxide, ferrosoferric oxide/black iron oxide, propylene glycol, and shellac glaze.

The article entitled “Extended-Release Once-Daily Formulation of Tofacitinib: Evaluation of Pharmacokinetics Compared With Immediate-Release Tofacitinib and Impact of Food” of authors Manisha Lamba et al published in Journal of clinical pharmacology 2016 November; 56(11): 1362-1371 discloses an extended-release (XR) tablet formulation of Tofacitinib has been developed using extrudable core system (ECS) osmotic delivery technology. Compared with conventional bilayer, push-pull osmotic tablets, the ECS tablet consists of a single-layer osmotically active core surrounded by a semipermeable membrane with a drug delivery port in the membrane. This technology has been shown to enhance the upper limit of drug loading in osmotic tablets. The ECS tablet formulation of Tofacitinib has been designed to achieve equivalence in total systemic exposure, as measured by area under the plasma concentration-time curve (AUC), relative to the IR formulation administered BID. In addition, the XR formulation was engineer to provide similarity in other PK parameters, including maximum (Cₘₐₓ) and minimum plasma concentration (Cₘᵢₙ) compared with the IR formulation.

The Chinese application CN110787145, discloses the sustained-release tablet of Tofacitinib citrate. The sustained-release tablet uses a sustained-release matrix tablet core and a sustained-release coating film that can make the drug release slowly over a period, can avoid the shortage of large fluctuations in blood concentration of ordinary tablets, can reduce the frequency of medication and can improve patient compliance.

The Chinese application CN108066319, discloses Tofacitinib citrate enteric-coated sustained-release pellet contains a matrix-type pellet core and an enteric coating coated on the pellet core. The matrix-type pellet core comprises matrix-type sustained-release material. The matrix-type sustained-release material elected from two or three of hydrophilic matrix materials, erodible matrix materials and non-erodible matrix materials.

The US Patent Application, US20140271842, discloses the extrudable osmotic tablet composition containing Tofacitinib citrate, sorbitol, hydroxyethyl cellulose, Kollidon-VA64, and magnesium stearate. It disclose a once daily pharmaceutical dosage form comprising a core Tofacitinib, or pharmaceutically acceptable salt thereof, and an osmogen, and a semipermeable membrane coating surrounding the core, wherein said coating comprises a water-insoluble polymer, wherein said dosage form is a sustained release dosage form, wherein the water-insoluble polymer is a cellulose derivative that sustains release of the Tofacitinib, or pharmaceutically acceptable salt thereof. The said patent discloses the tablet comprising Tofacitinib and polyethylene oxide; however said tablet is prepared by osmotic technology with drill hole at one end of tablet.

The PCT patent application WO2014174073 disclose sustained release formulations for oral administration comprising Tofacitinib or a pharmaceutically acceptable salt thereof, an alkalizing agent and hydrophilic polymer, wherein hydrophilic polymer is polyethylene oxide.

The PCT patent application WO2017029587 discloses sustained release oral pharmaceutical compositions of Tofacitinib. It contains release-controlling polymer and pharmaceutically acceptable excipients, wherein the sustained release oral pharmaceutical compositions further comprise an outer modified release coating that includes a modified release polymer. The invention also relates to a process for the preparation of the compositions. This patent application discloses the osmotic tablet comprising Tofacitinib and polyethylene oxide.

The commercial available product for Tofacitinib extended release tablet is in the form of osmotic delivery system. The said tablets having drilled hole at one end of the tablet. The tablets with osmotic drug delivery is costly, complex and also problems of Gastro intestinal obstruction, serious gastrointestinal reaction, Dose dumping, size of drilled hole is critical, Retrieval therapy is not possible in the case of unexpected adverse events. Also the product known in the prior art for Tofacitinib extended release tablet contain polyethylene oxide and they are in the form of osmotic drug delivery system and having drilled hole at one end of the tablet.

Therefore, there is need in the art to provide simple, cost effective, commercial feasible non osmotic extended release tablet of Tofacitinib and which also avoids, minimizes problems associated with osmotic drug delivery system like Gastro intestinal obstruction, serious gastrointestinal reaction, Dose dumping, drilled hole complexity.

Accordingly, applicant of the present invention invented novel non-osmotic extended release composition of tofacitinib or salt thereof comprising mixture of polyethylene oxide, which provides optimum drug release in the treatment of Rheumatoid Arthritis, Psoriatic Arthritis and Ulcerative Colitis. The novel extended release composition of tofacitinib or salt thereof comprising mixture of polyethylene oxide is simple, cost effective, commercial feasible and minimizes problems associated with osmotic drug delivery system like Gastro intestinal obstruction, serious gastrointestinal reaction, Dose dumping, drilled hole; therefore provides patient compliance in the treatment of Rheumatoid Arthritis, Psoriatic Arthritis and Ulcerative Colitis

### OBJECT OF THE INVENTION

Accordingly, it is an object of the present invention to provide novel extended release composition comprising Tofacitinib or salt thereof.

It is another object of the present invention to provide an extended release composition comprising Tofacitinib or salt thereof and mixture of polyethylene oxide.

It is another object of the present invention to provide an extended release composition comprising Tofacitinib or salt thereof and mixture of polyethylene oxide; wherein mixture contain polyethylene oxide with different molecular weight.

It is another object of the present invention to provide an extended release composition comprising Tofacitinib or salt thereof and mixture of polyethylene oxide; wherein mixture contain polyethylene oxide with molecular weight ranging from 20000 g/mol to 10000000 g/mol

It is another object of the present invention to provide an extended release composition comprising Tofacitinib or salt thereof and mixture of polyethylene oxide; wherein composition is not in the form of osmotic drug delivery system.

It is another object of the present invention to provide, an extended release composition comprising Tofacitinib or salt thereof, mixture of polyethylene oxide and one or more pharmaceutically acceptable excipient; wherein composition is not in the form of osmotic drug delivery system.

It is another object of the present invention to provide, process of manufacturing non-osmotic an extended release composition comprising Tofacitinib or salt thereof, mixture of polyethylene oxide and one or more pharmaceutically acceptable excipient.

It is another object of the present invention to provide, non-osmotic an extended release composition comprising Tofacitinib or salt thereof, mixture of polyethylene oxide and one or more pharmaceutically acceptable excipient in the treatment of Rheumatoid Arthritis, Psoriatic Arthritis and Ulcerative Colitis.

### SUMMARY OF THE INVENTION

The present invention provides an extended release composition of Tofacitinib and salt thereof, and process of manufacture thereof. The present invention particularly provides an extended release composition of Tofacitinib or salt thereof comprising mixture of polyethylene oxide. The extended release composition of Tofacitinib or salt thereof comprises mixture of polyethylene oxide with different molecular weight. The extended release composition of Tofacitinib or salt thereof comprises mixture of polyethylene oxide; wherein molecular weight of polyethylene oxide ranging from 20000 g/mol to 10000000 g/mol. The extended release composition of Tofacitinib or salt thereof comprising mixture of polyethylene oxide along with one or more pharmaceutically acceptable excipient; wherein composition is not in the form of osmotic drug delivery system. The non-osmotic extended release composition of Tofacitinib or salt thereof comprising mixture of polyethylene oxide along with one or more pharmaceutically acceptable excipient provides in vitro release profile of Tofacitinib similar to that of Xeljanz® XR commercially available tablet.

### DETAILED DESCRIPTION OF THE INVENTION

The present invention relates to extended release composition of Tofacitinib or salt thereof, and process of manufacture thereof.

The term “extended release composition” as used herein means a formulation in which the dissolution rate of the drug from the formulation is less than 85% after 30 minutes from the beginning a dissolution test. The dissolution test is carried out under in accordance with a dissolution test (paddle method) described in the United States Pharmacopoeia under the conditions that 900 mL of an appropriate test fluid (such as a USP buffer, pH 6.8) is used and the paddle rotation speed is 50 rpm.

The term “extended release” according to present invention can be used interchangeably with “sustained release”, “slow release”, “controlled release”, “modified release” or “long term release”.

The term composition or formulation according to present invention are similar and can be used interchangeably. The term composition according to present invention is intended to encompass at least one active ingredient, and at least one pharmaceutical acceptable excipient.

The active ingredient Tofacitinib may be used in the composition in its free base form or pharmaceutically acceptable salt. The Tofacitinib may be used in its acid addition salt. Examples of such a salt include an acid addition salt with citric acid, hydroiodic acid, nitric acid, hydrochloric acid, hydrobromic acid, sulfuric acid, phosphoric acid, formic acid, acetic acid, propionic acid, oxalic acid, malonic acid, succinic acid, fumaric acid, maleic acid, lactic acid, malic acid, tartaric acid, carbonic acid, picric acid, methanesulfonic acid, ethanesulfonic acid, glutamic acid or the like. Preferably, Tofacitinib is in the form of Tofacitinib citrate salt.

The extended release composition according to present invention contain active ingredient Tofacitinib is in the range of 0.1 mg to 50 mg; preferably equivalent to 11 mg and 22 mg.

In another embodiment of the present invention is to provide an extended release composition comprising Tofacitinib or salt thereof and mixture of polyethylene oxide.

Polyethylene oxide is hydrophilic rate controlling excipient used to extend or prolong the release of Tofacitinib from the composition. Polyethylene oxide polymer is hydrophilic, with high capacity to swell and erode in a controlled manner when exposed to dissolution media. Polyethylene oxide is polymer of ethylene oxide. Polyethylene oxide are polymers with a molecular mass above 20,000 g/mol. It is chemically similar to polyethylene glycol, but has a high molecular weight. Pharmaceutical grades of PEO are available commercially under the trade name of POLYOX™ water-soluble resins (WSR), manufactured by the Dow Wolff. They are novel materials with unique properties such as non-ionic, highly swelling, hydrophilic and thermoplastic behavior. PEO are safe, non-toxic polymers that are not absorbed through the gastro-intestinal tract.

An extended release composition of Tofacitinib or salt thereof according to present invention contain mixture of two or more polyethylene oxide with different grades and having different molecular weight. The molecular weight of polyethylene oxide ranges from 20000 g/mol to 10000000 g/mol. preferably molecular weight of polyethylene oxide ranges from 100000 g/mol to 8000000 g/mol. The different grades of polyethylene oxide available from Dow Wolff are WSR N-10NF (100000), WSR N-80F (200000), WSR N-750F (300000), WSR 205 NF (600000), WSR-1105NF (900000), WSR N-12K NF (1000000), WSR N-60K NF (2000000), WSR-301 NF (4000000), WSR coagulant NF (5000000), WSR-303 NF (7000000). The extended release composition comprises mixture of two-polyethylene oxide in the ratio ranging from 1:0.1 to 1:100 and vice versa; preferably the ratio ranging from 1:1 to 1:10 and vice versa.

In another embodiment of the present invention is to provide an extended release composition comprising Tofacitinib or salt thereof and mixture of polyethylene oxide; wherein composition is not in the form of osmotic drug delivery system.

Osmotic drug delivery devices are composed of an osmotically active drug core, which is surrounded by a rate controlling semipermeable membrane. Osmotic drug delivery system differ from diffusion based systems in the way of delivery of the active agents which is driven by an osmotic gradient somewhat than the concentration of drug in the device. In the most simple type of osmosis-controlled drug, release the following sequence of steps is involved in the release process:
1) Osmotic transport of liquid in to release unit.
2) Dissolution of drug within the release unit.
3) Convecting transport of a saturated drug solution by pumping of the solution through a single orifice through pores in the semi permeable membrane.

Osmotic delivery systems contain at least one delivery orifice in the semipermeable membrane for drug release and the size of delivery orifice must be optimized in order to control the drug release from osmotic systems. Osmogen are essential ingredient of the osmotic formulations. Upon penetration of biological fluid into the osmotic system through semipermeable membrane, osmogen are dissolved in the biological fluid, which creates osmotic pressure buildup inside the osmotic system and pushes medicament outside the dosage form through delivery orifice. Therefore extended release composition according to present invention is free from drug delivery orifice; also, it does not contain osmogen. An extended release composition according to present invention is in the form of extended matrix system; wherein extended release agent is polyethylene oxide and release of drug from the dosage form is via diffusion and erosion system.

In another embodiment of the present invention is to provide, non-osmotic an extended release composition comprising Tofacitinib or salt thereof, mixture of polyethylene oxide along with one or more pharmaceutically acceptable excipient

The term pharmaceutically acceptable excipient means a pharmacologically inactive component. The excipient(s) that are useful in preparing a pharmaceutical composition are generally safe, non-toxic and are acceptable for human as well as veterinary pharmaceutical use.

The one or more pharmaceutically acceptable excipient according to present invention may be selected from the group consisting of diluents, extended release agent, binders, lubricant, plasticizers, anti-tacking agents, opacifiers, coating polymers, solvents combination thereof.

The diluent include but not limited to lactose, microcrystalline cellulose, polyethylene glycol mannitol, sugar, dextrates, dextrin, dextrose, fructose, lactitol, sucrose, starch, xylitol, sorbitol, talc, calcium carbonate, calcium phosphate dibasic or tribasic, calcium sulphate, or combination thereof and alike. The most preferably diluents are Lactose, microcrystalline cellulose, polyethylene glycol. The composition according to present invention contains diluent from 10 to 80% by weight of composition.

The extended release agent according to present invention are polyethylene oxide. The extended release composition of Tofacitinib or salt thereof contain mixture of two or more polyethylene oxide with different molecular weight. Along with mixture of polyethylene oxide composition may contain extended release agent include but not limited to Methylcellulose, Hydroxypropylmethylcellulose, Hydroxypropylcellulose, Hydroxyethylcellulose, Ethylhydroxyethylcellulose, Sodium-carboxymethylcellulose, Sodium alginate, Xanthan gum, Carrageenan, Chitosan, Guar gum, Pectin, Ethylcellulose, Hypromellose acetate succinate, cellulose acetate, cellulose acetate propionate. Preferably, extended release agent is polyethylene oxide. The composition according to present invention contains extended release agent from 5 to 80% by weight of composition. Preferably, extended release agent are in the range of 20 to 60% by weight of composition.

The binder include but not limited to Povidone, starch; celluloses such as hydroxypropyl cellulose, hydroxyethyl cellulose, hydroxypropyl methylcellulose, ethyl cellulose, sodium carboxy methylcellulose; natural gums like acacia, alginic acid, guar gum; liquid glucose, dextrin, povidone, syrup, polyethylene oxide, poly-N-vinyl amide, polyethylene glycol, gelatin, poly propylene glycol, tragacanth or combination thereof and alike. Preferably, binder is povidone. The composition according to present invention contains binder from 0.5 to 10% by weight of composition.

The Lubricant include but not limited to magnesium, aluminum or calcium or zinc stearate, stearic acid, polyethylene glycol and talc or combination thereof. Preferably, lubricant is magnesium stearate. The composition according to present invention contains lubricant from 0.1 to 2% by weight of composition.

The plasticizer include but not limited to Polyethylene glycol, Propylene glycol, Triethyl citrate, triacetin, Waxes, or combination thereof.

The term “Anti-tacking Agent” is a necessary component in a coating system to prevent tackiness of the dosage forms during the manufacturing process. The anti-tacking agent(s) is selected form the group consisting of talc, silicon dioxide, simethicone, glycerol monosterate or combination thereof and alike. Preferably, anti-tacking agents are talc and silicon dioxide.

The term “Opacifier(s)” used to give more pastel color and increase film coverage. They can provide white coat or mask the color of the tablet/pellet/granule core. These are mostly inorganic material. Opacifier is titanium dioxide.

The coating polymer(s) selected from the group consisting of, polyvinyl alcohol, povidone or combination thereof and alike.

Solvents are chemical substances that can dissolve, suspend or extract other materials usually without chemically changing either the solvents or the other materials. Solvents can be organic or inorganic. They used to enhance solubility, taste, anti-microbial effectiveness or stability, to reduce dose volume or to optimize insolubility. Solvents also used to help the final product in achieving proper consistency. The solvent is selected form the group consisting of isopropyl alcohol, dichloromethane, Acetone and Purified water or combination thereof and alike.

In another embodiment of the present invention is to provide, non-osmotic an extended release composition comprising
i) Tofacitinib or salt thereof
ii) mixture of polyethylene oxide
iii) one or more other pharmaceutically acceptable excipient

In another embodiment of the present invention is to provide, non-osmotic an extended release composition comprising
i) Tofacitinib or salt thereof
ii) mixture of polyethylene oxide; wherein mixture comprises p polyethylene oxide with molecular weight ranges from 20000 g/mol to 10000000 g/mol
iii) one or more other pharmaceutically acceptable excipient

In another embodiment of the present invention is to provide, non-osmotic an extended release composition comprising
i) Tofacitinib or salt thereof
ii) mixture of polyethylene oxide; wherein mixture comprises polyethylene oxide with molecular weight ranges from 100000 g/mol to 8000000 g/mol
iii) one or more other pharmaceutically acceptable excipient

In another embodiment of the present invention is to provide, non-osmotic an extended release composition comprising
i) Tofacitinib or salt thereof
ii) mixture of polyethylene oxide; wherein concentration of polyethylene oxide ranges from 20 to 60 by weight of composition.
iii) one or more other pharmaceutically acceptable excipient

In another embodiment of the present invention is to provide, an extended release composition comprising
i) Tofacitinib or salt thereof
ii) Mixture of polyethylene oxide.
iii) one or more other pharmaceutically acceptable excipient
   wherein composition is free from osmogen.

In another embodiment of the present invention is to provide, non-osmotic an extended release composition comprising
i) Tofacitinib or salt thereof
ii) mixture of polyethylene oxide
iii) diluent
iv) optionally one or more other pharmaceutically acceptable excipient

In another embodiment of the present invention is to provide, non-osmotic an extended release composition comprising
i) Tofacitinib or salt thereof
ii) mixture of polyethylene oxide
iii) binder
iv) optionally one or more other pharmaceutically acceptable excipient

In another embodiment of the present invention is to provide, non-osmotic an extended release composition comprising
i) Tofacitinib or salt thereof
ii) mixture of polyethylene oxide
iii) diluent
iv) binder
v) optionally one or more other pharmaceutically acceptable excipient

In another embodiment of the present invention is to provide, non-osmotic an extended release composition comprising
i) Tofacitinib or salt thereof
ii) mixture of polyethylene oxide
iii) diluent selected from the group consisting of lactose, polyethylene glycol, microcrystalline cellulose or mixture thereof.
iv) optionally one or more other pharmaceutically acceptable excipient

In another embodiment of the present invention is to provide, non-osmotic an extended release composition comprising
i) Tofacitinib or salt thereof
ii) mixture of polyethylene oxide
iii) binder selected from the group consisting of povidone, hydroxyl propyl cellulose, starch or mixture thereof.
iv) optionally one or more other pharmaceutically acceptable excipient

In another embodiment of the present invention is to provide, non-osmotic an extended release composition comprising
i) Tofacitinib or salt thereof
ii) mixture of polyethylene oxide
iii) diluent selected from the group consisting of lactose, polyethylene glycol, microcrystalline cellulose or mixture thereof.
iv) binder selected from the group consisting of povidone, hydroxyl propyl cellulose, starch or mixture thereof.
v) optionally one or more other pharmaceutically acceptable excipient

The non-osmotic an extended release composition according to present invention may be in the form of tablet, capsule, sachet, granules, beads, pellets or powder.

In another embodiment of the present invention is to provide, process of manufacturing non-osmotic an extended release composition comprising Tofacitinib or salt thereof, mixture of polyethylene oxide and one or more pharmaceutically acceptable excipient.

The process of manufacturing non-osmotic an extended release composition comprises the step of
i) Dry Mix Tofacitinib or salt thereof, polyethylene oxide along with one or more excipient
ii) Dissolve binder in to the solvent to form binder solution
iii) Add binder solution of step ii) in to the dry mixt of step i) to form the wet mass
iv) Dry the wet mass and Sieve to form the granules
v) Mix the granules in step iv) with lubricant
vi) Compress the mixture of step v) to form the tablet or fill the mixture in to the capsule or sachet

The process of manufacturing non-osmotic an extended release composition comprises the step of
i) Dry Mix Tofacitinib or salt thereof, polyethylene oxide, lactose, polyethylene glycol
ii) Dissolve povidone in to the isopropyl alcohol to form binder solution
iii) Add binder solution of step ii) in to the dry mixt of step i) to form the wet mass.
iv) Dry the wet mass and Sieve to form the granules.
v) Mix the granules in step iv) with magnesium stearate.
vi) Compress the mixture of step v) to form the tablet or fill the mixture in to the capsule or sachet

In another embodiment of the present invention is to provide, process of manufacturing non-osmotic an extended release composition comprises the step of
i) Dry Mix Tofacitinib or salt thereof, polyethylene oxide, lactose, polyethylene glycol.
ii) Add the magnesium stearate in to the dry mix of step i)
iii) Compress the mixture of step ii) to form the tablet or fill the mixture in to the capsule or sachet

The extended release composition of Tofacitinib or salt thereof according to present invention; wherein manufacturing process of composition involves dry granulation method or wet granulation method or extrusion-spheronization method.

The process of manufacturing, concentration of Tofacitinib or salt thereof, concentration of one or more pharmaceutically acceptable excipient according to present invention optimized in such way that, composition provides optimum release of Tofacitinib in the treatment of Rheumatoid Arthritis, Psoriatic Arthritis and Ulcerative Colitis.

In another embodiment of the present invention is to provide, non-osmotic an extended release composition comprising Tofacitinib or salt thereof, mixture of polyethylene oxide and one or more pharmaceutically acceptable excipient; wherein dissolution of Tofacitinib from the composition is

a) not more than 20% in 1 hour

b) not less than 50% and not more than 80% in 3 hours and

c) not less than 80% in 6 hours

When measured in-vitro in USP Apparatus Type 2 (Paddle) using pH 6.8-phosphate buffer of 900 mL, at 50 rpm. The said composition is preferably in the form of tablet, tablet in capsule, capsule or pellet in capsule.

The non-osmotic an extended release composition of Tofacitinib or salt thereof comprising mixture of polyethylene oxide and one or more pharmaceutically acceptable excipient provides similar in-vitro drug release profile as that of commercially available Xeljanz® extended release tablet. Therefore, composition according to present invention is found to be in compliance

The non-osmotic extended release composition of tofacitinib or salt thereof comprising mixture of polyethylene oxide along with one or more pharmaceutically acceptable excipient achieves desired drug release profile in the treatment of Rheumatoid Arthritis, Psoriatic Arthritis and Ulcerative Colitis. The composition according to present invention is simple, cost effective, commercially feasible and minimizes problems associated with osmotic drug delivery system like Gastro intestinal obstruction, serious gastrointestinal reaction, Dose dumping, drilled hole complexity; therefore provides patient compliance in the treatment of Rheumatoid Arthritis, Psoriatic Arthritis and Ulcerative Colitis.

The non-osmotic an extended release composition of Tofacitinib or salt thereof prepared with mixture of polyethylene oxide with different molecular weight compared with extended release composition of Tofacitinib or salt thereof prepared with single polyethylene oxide. However, it was found that composition with single polyethylene oxide does not provide desired dissolution profile when compared with reference product Xeljanz XR and. The composition of Tofacitinib or salt thereof prepared with mixture of polyethylene oxide provides optimum and desired release profile when compared with reference product Xeljanz XR and in the treatment of Rheumatoid Arthritis, Psoriatic Arthritis and Ulcerative Colitis.

Therefore non-osmotic an extended release composition of Tofacitinib or salt thereof according to present invention contain mixture of polyethylene oxide grades as single or standalone use of polyethylene oxide does not offer adequate release profile. In addition, the composition according to present invention is free from osmogen.

In another embodiment of the present invention is to provide the stable non-osmotic an extended release composition of Tofacitinib or salt thereof comprising mixture of polyethylene oxide and one or more pharmaceutically acceptable excipient. The extended release composition of Tofacitinib or salt according to present invention were loaded for stability study at condition of 40° C./75% RH, 30° C./75% RH, 25° C./60% RH as per ICH guideline. After stability study, in-vitro drug release profile, assay, related substances and other parameters found to be in the compliance; therefore, composition according to the invention is found to be stable.

The non-osmotic an extended release composition of Tofacitinib or salt thereof according to present invention packaged in suitable airtight containers and moisture proof packs. The pharmaceutical composition of the present invention preferably packaged in to the strip, blister, bottle or sachet

### EXAMPLE

The following Examples are provided solely for illustrative purposes and are not meant to limit the invention in any way. Some illustrative non-limiting examples of the present invention as described below.

TABLE 1
Example-1

| Ingredients | Quantity (mg) |
|---|---|
| Tofacitinib Citrate | 17.77* |
| Lactose Monohydrate | 53.23 |
| Polyethylene oxide 900000 | 80.00 |
| Microcrystalline cellulose | 20.00 |
| Binder | |
| Povidone | 8.00 |
| Solvent | Qs |
| Lubrication | |
| Magnesium Stearate | 1.00 |
| Total | 180.00 |
| Film coating | |
| Opadry II Pink 85F540244 | 5.00 |
| (PVA, TiO2, Talc, PEG, | |
| Red oxide of iron & | |
| Black oxide of iron) | |
| Solvent | Qs |
| Total | 185.00 |

*17.77 mg Tofacitinib Citrate eq. to 11 mg of Tofacitinib

### Manufacturing Procedure:

1. Dry mix Tofacitinib citrate, lactose, polyethylene oxide & microcrystalline cellulose in a high shear mixer
2. Add binder and knead the wet mass
3. Dry the wet mass and sieve the granules
4. Add magnesium stearate in a low shear mixer
5. Compress the granules
6. Coat the tablets

TABLE 2
Example-2

| Ingredients | Quantity (mg) |
|---|---|
| Tofacitinib Citrate | 17.77* |
| Mannitol | 20.00 |
| Lactose Monohydrate | 53.23 |
| Polyethylene oxide 900000 | 90.00 |
| Binder | |
| Povidone | 8.00 |
| Solvent | Qs |
| Lubrication | |
| Magnesium Stearate | 1.00 |
| Total | 190.00 |
| Film coating | |
| Opadry II Pink 85F540244 | 5.00 |
| (PVA, TiO2, Talc, PEG, | |
| Red oxide of iron & | |
| Black oxide of iron) | |
| Solvent | Qs |
| Total | 195.00 |

*17.77 mg Tofacitinib Citrate eq. to 11 mg of Tofacitinib

### Manufacturing Procedure

1. Dry mix Tofacitinib citrate, mannitol, lactose, polyethylene oxide in a high shear mixer
2. Add binder and knead the wet mass
3. Dry the wet mass and sieve the granules
4. Add, magnesium stearate in a low shear mixer
5. Compress the granules
6. Coat the tablets

TABLE 3
Example-3

| Ingredients | Quantity (mg) |
|---|---|
| Tofacitinib Citrate | 17.77* |
| Microcrystalline Cellulose | 20.00 |
| Polyethylene oxide 7000000 | 140.00 |
| Binder | |
| Povidone | 15.00 |
| Solvent | Qs |
| Lubrication | |
| Magnesium Stearate | 3.00 |
| Total | 196.00 |
| Film Coating | |
| Opadry II Pink 85F540244 | 5.00 |
| (PVA, TiO2, Talc, PEG, | |
| Red oxide of iron & | |
| Black oxide of iron) | |
| Solvent | Qs |
| Total | 201.00 |

*17.77 mg Tofacitinib Citrate eq. to 11 mg of Tofacitinib

### Manufacturing Procedure:

1. Dry mix Tofacitinib citrate, microcrystalline cellulose, polyethylene oxide in a high shear mixer
2. Add binder and knead the wet mass
3. Dry the wet mass and sieve the granules
4. Add magnesium stearate in a low shear mixer
5. Compress the granules
6. Coat the tablets

TABLE 4
Example-4

| Ingredients | Quantity (mg) |
|---|---|
| Tofacitinib citrate | 17.77* |
| Lactose Monohydrate | 53.23 |
| Polyethylene oxide 900000 | 20.00 |
| Polyethylene oxide 2000000 | 80.00 |
| PEG 6000 | 20.00 |
| Binder | |
| PVPK 30 | 8.00 |
| IPA | |
| Lubrication | |
| Magnesium Stearate | 1.00 |
| Total | 200.00 |
| Film Coating | |
| Opadry II Pink 85F540244 | 5.00 |
| (PVA, TiO2, Talc, PEG, | |
| Red oxide of iron & | |
| Black oxide of iron) | |
| Purified Water | Qs |
| Total | 205.00 |

*17.77 mg Tofacitinib Citrate eq. to 11 mg of Tofacitinib

Manufacturing procedure:
1. Dry mix Tofacitinib citrate, lactose, polyethylene oxides in a high shear mixer
2. Add binder and knead the wet mass
3. Dry the wet mass and sieve the granules
4. Add magnesium stearate in a low shear mixer
5. Compress the granules
6. Coat the tablets

TABLE 5
Example-5

| Ingredients | Quantity (mg) |
|---|---|
| Tofacitinib citrate | 17.77* |
| Lactose Monohydrate | 53.23 |
| Polyethylene oxide 900000 | 20.00 |
| Polyethylene oxide 2000000 | 100.00 |
| Polyethylene glycol 6000 | 20.00 |
| Binder | |
| Povidone | 8.00 |
| Lubrication | |
| Magnesium Stearate | 1.00 |
| Total | 200.00 |
| Film Coating | |
| Opadry II Pink 85F540244 | 10.00 |
| (PVA, TiO2, Talc, PEG, | |
| Red oxide of iron & | |
| Black oxide of iron) | |
| Solvent | Qs |
| Total | 210.00 |

*17.77 mg Tofacitinib Citrate eq. to 11 mg of Tofacitinib

Manufacturing procedure:
1. Dry mix Lactose, polyethylene oxide, polyethylene glycol & Povidone in a low shear mixer
2. Add magnesium stearate in a low shear mixer
3. Compress the granules
4. Coat the tablets

### Dissolution Data:

The in-vitro release profile of the examples 1-5 has been performed in the medium: pH 6.8 Phosphate buffer, Apparatus: USP Type 2 (Paddle with Sinker), 900 ml, 50 rpm.

The example 1 and 2, as those having only one PEO, lack the controlled release profile whereas the example 4-5, wherein the composition comprises the mixture of two PEO provide the controlled release profile.

TABLE 6
Dissolution Data for Examples 1 to 5 and Reference product

| Time | Exam- | Exam- | Exam- | Exam- | Exam- | Reference |
|---|---|---|---|---|---|---|
| (Hour) | ple 1 | ple 2 | ple 3 | ple 4 | ple 5 | product |
| 1 | 40 | 36 | 9 | 10 | 12 | 6 |
| 1.5 | 75 | 73 | 12 | 20 | 17 | 16 |
| 2 | 90 | 88 | 15 | 32 | 33 | 31 |
| 2.5 | 97 | 92 | 19 | 45 | 57 | 50 |
| 3 | 99 | 95 | 22 | 60 | 68 | 63 |
| 4 | 99 | 97 | 28 | 75 | 81 | 77 |
| 6 | 99 | 97 | 39 | 92 | 96 | 95 |
| F2 | 19 | 20 | 23 | 73 | 68 | |
| Value | | | | | | |

The release profile of the examples 4-5 are comparable, even better, to that of the release profile of the reference product.

Stability Data:

TABLE 7
Test Product; Stability condition 40° C./75% RH

| | | Dissolution (%) | | | | | | | |
|---|---|---|---|---|---|---|---|---|---|
| | | Media: pH 6.8 | | | Related Substances | | | | |
| | | Phosphate buffer, 900 ml, | | | | | | Highest | |
| | Assay | Paddle with sinker, 50 RPM | | | Acetyl | Chloroacetyl | | Unknown | Total |
| | Limit: | NMT | 55- | NLT | Impurity | Impurity | Diasteromer | Impurity | Impurities |
| Tests | 95.0- | 45% | 80% | 85% | NMT | NMT | NMT | NMT | NMT |
| Condition | 110.0% | 2 hrs | 4 hrs | 8 hrs | 0.3% | 0.3% | 0.3% | 0.2% | 2.0% |
| Initial | 98.3 | 38 | 75 | 101 | BDL | ND | ND | BDL | BDL |
| 1M (40/75) | 96.8 | 31 | 63 | 95 | BDL | ND | ND | BDL | BDL |
| 2M (40/75) | 98.2 | 30 | 61 | 95 | BDL | ND | ND | BDL | BDL |
| 3M (40/75) | 97.0 | 32 | 64 | 94 | BDL | ND | ND | BDL | BDL |
| 6M (40/75) | 97.3 | 34 | 67 | 95 | BDL | ND | ND | BDL | BDL |

BDL # below detectable limit
ND # not detectable

TABLE 8
Test Product; Stability condition 30° C./75% RH

| | | Dissolution (%) | | | | | | | |
|---|---|---|---|---|---|---|---|---|---|
| | | Media: pH 6.8 | | | Related Substances | | | | |
| | | Phosphate buffer, 900 ml, | | | | | | Highest | |
| | Assay | Paddle with sinker, 50 RPM | | | Acetyl | Chloroacetyl | | Unknown | Total |
| | Limit: | NMT | 55- | NLT | Impurity | Impurity | Diasteromer | Impurity | Impurities |
| Tests | 95.0- | 45% | 80% | 85% | NMT | NMT | NMT | NMT | NMT |
| Condition | 110.0% | 2 hrs | 4 hrs | 8 hrs | 0.3% | 0.3% | 0.3% | 0.2% | 2.0% |
| Initial | 98.3 | 38 | 75 | 101 | BDL | ND | ND | BDL | BDL |
| 3M (30/75) | 97.0 | 32 | 64 | 94 | BDL | ND | ND | BDL | BDL |
| 6M (30/75) | 97.9 | 33 | 66 | 99 | BDL | ND | ND | BDL | BDL |

BDL # below detectable limit
ND # not detectable

TABLE 9
Test Product; stability condition 25° C./60% RH

| | | Dissolution (%) | | | | | | | |
|---|---|---|---|---|---|---|---|---|---|
| | | Media: pH 6.8 | | | Related Substances | | | | |
| | | Phosphate buffer, 900 ml, | | | | | | Highest | |
| | Assay | Paddle with sinker, 50 RPM | | | Acetyl | Chloroacetyl | | Unknown | Total |
| | Limit: | NMT | 55- | NLT | Impurity | Impurity | Diasteromer | Impurity | Impurities |
| Tests | 95.0- | 45% | 80% | 85% | NMT | NMT | NMT | NMT | NMT |
| Condition | 110.0% | 2 hrs | 4 hrs | 8 hrs | 0.3% | 0.3% | 0.3% | 0.2% | 2.0% |
| Initial | 98.3 | 38 | 75 | 101 | BDL | ND | ND | BDL | BDL |
| 3M (25/60) | 97.5 | 33 | 63 | 93 | BDL | ND | ND | BDL | BDL |
| 6M (25/60) | 98.2 | 31 | 62 | 94 | BDL | ND | ND | BDL | BDL |

BDL # below detectable limit
ND # not detectable

1. An extended release composition comprising
a) Tofacitinib or salt thereof,
b) mixture of two or more polyethylene oxide and
c) Optionally one or more pharmaceutically acceptable excipient.

2. The extended release composition as claimed in claim 1, wherein composition is not in the form of osmotic drug delivery system.

3. The extended release composition as claimed in claim 1, wherein amount of Tofacitinib is equivalent to 11 mg or 22 mg.

4. The extended release composition as claimed in claim 1, wherein molecular weight of polyethylene oxide ranges from 20000 g/mol to 10000000 g/mol.

5. The extended release composition as claimed in claim 1, wherein composition comprises mixture of two polyethylene oxide in the ratio ranging from 1:0.1 to 1:100

6. The extended release composition as claimed in claim 1, wherein composition is in the form of tablet, capsule, sachet, granules, beads, pellets or powder.

7. The extended release composition as claimed in claim 1, wherein dissolution of Tofacitinib from the composition is
a) not more than 20% in 1 hour
b) not less than 50% and not more than 80% in 3 hours and
c) not less than 80% in 6 hours
when measured in-vitro in USP Apparatus Type 2 (Paddle) using pH 6.8-phosphate buffer of 900 mL, at 50 rpm and said composition is in the form of tablet or tablet in capsule or capsule or pellet in capsule.

8. The extended release composition as claimed in claim 1, wherein one or more pharmaceutically acceptable excipient selected from the group consisting of diluent, binder, solvent and lubricating agent

9. The extended release composition as claimed in claim 8, wherein diluent is selected from the lactose, microcrystalline cellulose, mannitol or mixture thereof; binder is selected from the povidone or Hydroxy propyl cellulose or mixture thereof; solvent is selected from isopropyl alcohol, water or mixture thereof; lubricating agent is magnesium stearate.

10. The extended release composition as claimed in claim 1, wherein manufacturing process of composition involves dry granulation method or wet granulation method or extrusion-spheronization method.
